SEQUENCE LISTING (7 sequences)

SEQ ID NO: 1 - DNA, 19 bp, synthetic construct
  misc_feature 1..19: note = mouse siTACE Sense sequence
  source 1..19: mol_type = other DNA
acacctgctgcaatagtga

SEQ ID NO: 2 - DNA, 21 bp, synthetic construct
  misc_feature 1..21: note = human siTACE Sense sequence
  source 1..21: mol_type = other DNA
gctctcagactacgatattct

SEQ ID NO: 3 - DNA, 20 bp, synthetic construct
  misc_feature 1..20: note = mouse siTACE antisense sequence
  source 1..20: mol_type = other DNA
tcactattgcagcaggtgtt

SEQ ID NO: 4 - DNA, 21 bp, synthetic construct
  misc_feature 1..21: note = human siTACE antisense sequence
  source 1..21: mol_type = other DNA
agaatatcgtagtctgagagc

SEQ ID NO: 5 - protein, 15 aa, synthetic construct
  REGION 1..15: note = Cys-TKPR-(9Arg)-Cys
CTKPRRRRRRRRRRC

SEQ ID NO: 6 - DNA, 20 bp, synthetic construct
  misc_feature 1..20: note = mouse TACE Forward primer
  source 1..20: mol_type = other DNA
gtacgtcgatgcagagcaaa

SEQ ID NO: 7 - DNA, 20 bp, synthetic construct
  misc_feature 1..20: note = mouse TACE Reverse primer
  source 1..20: mol_type = other DNA
aaaccagaacagacccaacg

(19) United States
(12) Patent Application Publication
(10) Pub. No.: US 2024/0158798 A1
(43) Pub. Date: May 16, 2024

(54) NON-VIRAL GENE/CARRIER COMPLEX FOR PREVENTION OR TREATMENT OF ACUTE INFLAMMATORY DISEASE

(71) Applicants: IUCF-HYU (INDUSTRY-UNIVERSITY COOPERATION FOUNDATION HANYANG UNIVERSITY), Seoul (KR); INDUSTRY-UNIVERSITY COOPERATION FOUNDATION HANYANG UNIVERSITY ERICA CAMPUS, Ansan-si, Gyeonggi-do (KR)

(72) Inventors: Yong-Hee KIM, Seoul (KR); Ji Eun LEE, Seoul (KR); Ju Hyeong HONG, Seoul (KR); Chul-Su YANG, Ansan-si, Gyeonggi-do (KR)

(21) Appl. No.: 18/531,662

(22) Filed: Dec. 6, 2023

Related U.S. Application Data
(63) Continuation of application No. PCT/KR2022/007935, filed on Jun. 3, 2022.

(30) Foreign Application Priority Data
Jun. 7, 2021 (KR) 10-2021-0073528

(51) Int. Cl. C12N 15/113; A61K 47/64; A61P 37/06

(52) U.S. Cl. CPC C12N 15/1137; A61K 47/6455; A61P 37/06; C12N 2310/14

(57) ABSTRACT

The present invention relates to a gene/carrier complex having the effect of preventing or treating acute inflammatory disease, comprising a tumor necrosis factor-alpha converting enzyme (TACE) siRNA and a non-viral gene carrier, wherein the non-viral gene carrier comprises a TFA salt of TKPR-oligo-arginine.

### INCORPORATION OF SEQUENCE LISTING

This application contains a sequence listing submitted in Computer Readable Form (CRF). The CRF file containing the sequence listing entitled “9-PK003806438-SequenceListing.xml”, which was created on Dec. 6, 2023, and is 9,880 bytes in size. The information in the sequence listing is incorporated herein by reference in its entirety.

### TECHNICAL FIELD

The present invention relates to a non-viral gene/carrier complex for preventing or treating an acute inflammatory disease.

### BACKGROUND ART

Inflammation is an innate response to a pathogen or tissue damage caused by a pathogen, and when an inflammatory response is not controlled, harmful effects to a host can become very serious, so more thorough research is needed.

Acute inflammation causes symptoms such as pain, fever, flushing, and abscesses due to the invasion of external infection sources (bacteria, fungi, viruses, and various types of allergens). Generally, an inflammatory response induced when a stimulus that causes a certain substantial change is applied to cells or tissue of the living body is a defense system for protecting the living body.

Treatment of such inflammation may be promoted by phagocytosis, which is an action that suppresses the proliferation of invading bacteria or activates macrophages phagocytizing foreign substances, thereby enhancing the functions of macrophages for digesting and excreting the foreign substances. However, acute inflammatory responses such as sepsis and endotoxemia occur due to excessive activation of macrophages caused by bacterial by-products such as lipopolysaccharide (LPS). Excessive activation of macrophages leads to a subsequent increase in cytokines such as interleukins (Ills) and tumor necrosis factors (TNFs), ultimately leading to organ dysfunction.

The systemic inflammatory responses such as sepsis and endotoxemia present a significant clinical problem because the mortality of these diseases is 30 to 60%. The main cause of the high mortality is the result of dysfunction of multiple organs and subsequent organ failures. Therefore, anti-inflammatory drugs and treatments that can treat acute inflammatory diseases are urgently needed.

The tumor necrosis factor-α converting enzyme (TACE) shRNA (hereinafter, named “shTACE”) therapeutic gene itself according to the literature entitled “Research on the development of non-viral TACE interfering gene delivery system for the treatment of inflammatory disease (Somi Kim, Hanyang University Graduate School Master's thesis (February, 2014)),” which has been reported previously by the present applicant, has low stability in the human body, so its application to the human body is limited. In addition, since the action of shRNA occurs after passing through the nuclear membrane, the time to treat an acute inflammatory response may be missed, so it is necessary to deliver siRNA capable of RNA interference in the cytoplasm. One thing that should be overcome in gene therapy is that a gene therapeutic agent passes through the cell membrane to achieve efficient gene delivery. Accordingly, in gene therapy, research on a system for efficiently delivering an siRNA gene is required.

Moreover, in the case of existing delivery vehicles, cell-specific target delivery was difficult.

### DISCLOSURE

### Technical Problem

Therefore, the present inventors developed a non-viral gene/carrier complex, which targets acute inflammatory macrophages, by preparing a new gene therapeutic agent that modifies the sequence of siRNA suppressing the expression of an existing tumor necrosis factor (TNF)-α converting enzyme (TACE) and synthesizing a new gene carrier for stably delivering the gene therapeutic agent. Thus, the present invention was completed.

Accordingly, the present invention is directed to providing a gene/carrier complex, which includes siRNA suppressing TACE expression and a non-viral gene carrier.

In addition, the present invention is directed to providing a composition for preventing or treating an acute inflammatory disease, which includes the gene/carrier complex as an active ingredient.

In addition, the present invention is directed to providing a method of preventing or treating an acute inflammatory disease, which includes administering a therapeutically effective amount of composition including the gene/carrier complex to a subject.

### Technical Solution

The present invention relates to a gene/carrier complex, which includes siRNA suppressing TACE and a non-viral gene carrier.

### Advantageous Effects

In various acute inflammatory diseases such as acute sepsis, acute lung injury, acute liver failure, and acute inflammatory bowel disease, the invasion of macrophages into an organ with any one of the diseases increases, and here, the expression of tumor necrosis factor-α converting enzyme (TACE) is increased.

When administering the gene/carrier complex according to the present invention, treatment is possibly by targeting overexpressed inflammatory macrophages in the corresponding organ with the disease.

Particularly, the gene/carrier complex according to the present invention can be administered when a precise diagnosis cannot be made in acute inflammation caused by the invasion of microorganisms, bacteria, viruses, etc., or when an immediate anti-inflammatory effect is expected. In the case of existing shTACE, it takes time for a gene to be expressed after passing through both the cell membrane and the nuclear membrane, but in the case of siTACE, an anti-inflammatory effect can be expected through immediate mRNA interference after cell membrane penetration.

### DESCRIPTION OF DRAWINGS

FIGS. 1A-1E show the physicochemical characterization of an siTACE/TKPR-9R complex [FIG. 1A: Gel retardation assay. siTACE was coupled to various weight ratios of a TKPR-9R peptide-based carrier by incubation at room temperature for 30 minutes; FIG. 1B: Surface zeta potential, FIG. 1C: The mean diameter of the siTACE/TKPR-9R complex measured by dynamic light scattering; and FIG. 1D: The survival rate of the cells was measured by MTT assay after treatment of Raw 264.7 cells (n=8) with the siTACE/TKPR-9R complexes (weight ratio of TKPR-9R/siRNA=1, 2, 3, 4). The data is expressed as mean±S·D. In FIGS. 1B, IC, and ID, ‘+’ in the 3rd row indicates 1 μg, and ‘++’ and ‘+++’ indicate 1.5 μg and 2 μg of TKPR-9R, respectively; and for siTACE, +(1 μg), ++(2 μg), and +++(3 μg), and for TKPR, +(4 μg), ++(8 μg), and +++(12 μg). FIG. 1E: In vitro macrophage-targeting effect study of TKPR-9R. Competition assay in LPS-induced inflammatory Raw 264.7 macrophages between free TKPR-9R and an siRNA/TKPR-9R complex (weight ratio of TKPR-9R/siRNA=4) was performed by flow cytometry using FAM-conjugated siRNA].

FIGS. 2A-2D show the In vitro anti-inflammatory effects of siTACE/TKPR-9R complexes [After activating lipopolysaccharide (LPS) in macrophages, the cells were transfected with an siTACE/TKPR-9R complex (weight ratio of TKPR-9R/siRNA=4) for 24 hours.

FIG. 2A: The relative Tace mRNA level compared to GAPDH was measured by quantitative real-time PCR (qRT-PCR) and normalized to anon-treated LPS group (n=10). FIG. 2B: Downregulation of pro-inflammatory cytokine TNF-α protein level, FIG. 2C: IL-6 protein level, and FIG. 2D: chemokine MCP-1 protein levels secreted from the treated cell medium of each group were quantified using ELISA (n=8). The data is expressed as mean±S·D. In the 2nd row, ‘+’ indicates 1 μg, and ‘++’ and ‘+++’ indicate 2 μg and 3 μg of siTACE in the complexes, and in the 4th row, ‘+’ indicates 4 μg, and ‘++’ and “++++’ indicate 8 μg and 12 μg of TKPR-9R in the complexes and B, C, and D, respectively. Statistical differences compared to the LPS group were calculated using one-way ANOVA with Dunnett's test, *P<0.05, ** P<0.01, *** P<0.001, and n.s=not significant in A, B, C, or D].

FIGS. 3A-3F show in vivo combination therapy for systemic sepsis using siTACE/TKPR-9R and antibiotics [FIG. 3A: Cecal ligation and puncture (CLP)-induced sepsis model, I.V. Injection of siTACE/TKPR-9R complexes (siTACE gene: 1 mg/kg) and I.P. Injection of gentamicin (8 mg/kg) and cephalosporin (8 mg/kg). FIG. 3B: The survival rate of CLP-induced septic mice in each group was monitored for 5 days (n=15 and 16). Statistical differences were indicated compared to the CLP+PBS mice (long-rank test). Only one of 15 subjects lived and 14 subjects died over 4 days in the CLP+PBS group. FIG. 3C: Hematoxylin and eosin (H&E) staining and histopathology scores of lung and liver tissue sections on day 1 after treatment of siTACE/TKPR-9R complexes in septic mice. FIG. 3D: A pathologist graded histopathology scores and inflammation severity (scale bar: lung=500 μm, liver=200 μm). (FIG. 3E) E. coli and (FIG. 3F) P. aeruginosa were injected into C57BL/6 mice, siTACE/TKPR-9R (siTACE: 1 mg/kg, IV) and antibiotics (Gentamycin 8 mg/kg, IP and Cephalosporin 8 mg/kg, IP) were injected. siTACE/TKPR-9R complexes and antibiotics were injected once into septic mice in E and two antibiotics were injected twice in F. The survival rate of bacteria-induced septic mice in each group was monitored for 5 and 10 days (n=8). Statistical differences were indicated compared to PBS-treated mice (long-rank test)].

FIGS. 4A-4E show the ex vivo anti-inflammatory effects of siTACE/TKPR-9R complexes in tissue and serum [FIG. 4A: down-regulation of TACE protein expression level in tissue homogenates of lung, spleen, kidney, and liver. FIGS. 4A, 4B, and 4C: After CLP surgery, septic mice were treated with siTACE/TKPR-9R complexes (siTACE gene: 1 mg/kg, I.V.). FIG. 4A: A TACE protein level and FIG. 4B: A Tnf-α mRNA level were measured using actin and GAPDH as a control (n=4). Statistical differences compared to the PBS group were calculated using a two-tailed Student's test. FIG. 4C: Pro-inflammatory cytokines (TNF-α, IL-1β, and IL-6) and chemokine (MCP-1) levels from serum were measured by ELISA (n=6). Statistical differences compared to the PBS group were calculated using one-way ANOVA with Dunnett's test (*P<0.05, ** P<0.01, *** P<0.001, and n.s=not significant). FIGS. 4D and 4E: After administering E. coli and P. aeruginosa to the mice, siTACE/TKPR-9R (siTACE: 1 mg/kg, I.V.) and antibiotics (Gentamycin: 8 mg/kg, I.P., Cephalosporin: 8 mg/kg, I.P.) were used as therapeutic agents. FIG. 4D: Inflammatory cytokine level in serum (n=7). Downregulation of pro-inflammatory cytokine TNF-α protein level and IL-6 protein level, and IL-1β protein level in serum was quantified using ELISA. The data is expressed as mean±S·D (*P<0.05, ** P<0.01, and *** P<0.001). Statistical differences compared to the PBS group were calculated using one-way ANOVA with Dunnett's test (*P<0.05, ** P<0.01, *** P<0.001, and n.s=not significant). FIG. 4E: Bacterial loads in serum (left) and peritoneal fluid (right) were measured. The antibacterial effect of antibiotics was maintained against gram-negative bacteria E. coli DH5a on LB plates even when administered with siTACE/TKPR-9R complexes (n=7). Living bacterial colonies were counted. Statistical differences compared to the PBS group were calculated using a two-tailed Student's test].

FIGS. 5A-5C show a macrophage-targeting effect of siTACE/TKPR-9R complexes [FIG. 5A: in vivo biodistribution image. Normal mice and CLP-induced septic mice were intravenously injected with siRNA-Cy5/TKPR-9R complexes and then sacrificed at 1 h, 6 h, and 24 h after injection. The fluorescence intensity and relative mean fluorescence intensity (MFI) values in primary macrophages (f4/80+)(FIG. 5B) and non-macrophages (f4/80−)(FIG. 5C). Macrophages positive for F4/80 in peritoneal cavity show high cellular uptake of siRNA-FAM/TKPR-9R complexes (n=3)].

FIG. 6 shows an In vitro macrophage-targeting effect, which is a result for a human THP-1 uptake test. Competition assay in LPS-induced inflammatory THP-1 macrophages between free TKPR-9R and an siRNA/TKPR-9R complex (weight ratio of TKPR-9R/siRNA=4) was performed by flow cytometry using FAM-conjugated siRNA.

FIG. 7 shows the In vitro anti-inflammatory effect of human siTACE/TKPR-9R complexes [After LPS activation in THP-1 macrophages, cells were transfected with free human siTACE, human siTACE/PEI complexes, and a human siTACE/TKPR-9R complex (weight ratio of TKPR-9R/siRNA=4) for 24 hours. Relative Tace, Tnf-α, and I1-10 mRNA levels compared to GAPDH measured by quantitative real-time PCR (qRT-PCR) and normalized to a non-treated LPS group (n=5).

### MODES OF THE INVENTION

Hereinafter, a detailed description of the present invention is as follows.

For rapid inhibition of an inflammatory response, instead of a slowly expressing shRNA form, the present inventors developed siRNA (hereinafter, referred to as siTACE) that suppresses the expression of new tumor necrosis factor-α converting enzyme (TACE) and is capable of directly acting in the cytoplasm of a cell. For rapid expression, although existing shTACE should be continuously added to the cytoplasm as much as desired, the novel siTACE does not need to be added in large amounts, thereby having an economical advantage compared to existing gene therapeutic agents. Accordingly, for rapid inhibition of TCE in the cytoplasm, anti-inflammation may be induced within a short period of time using siRNA (i.e., siTACE).

In the present invention, siRNA against TACE may be nucleotides having one or more base sequences represented by SEQ ID NOs: 1 or 2 and a complementary sequence thereof.

Mouse siTACE

Sense sequence: SEQ ID NO: 1

Antisense sequence: SEQ ID NO: 3

Human siTACE

Sense sequence: SEQ ID NO: 2

Antisense sequence: SEQ ID NO: 4

siRNA is a double-stranded RNA form composed of sense/anti-sense RNAs, and may be expressed from a recombinant cyclic or linear DNA plasmid using any suitable promoter. Suitable promoters for expressing siRNA of the present invention from plasmids include, for example, a U6 or H1 RNA pol III promoter sequence and a cytomegalovirus promoter. Other suitable promoters may be selected by those of ordinary skill in the art. In addition, the recombinant plasmid of the present invention may include an inducible or regulatory promoter for expressing siRNA in a specific tissue or intracellular environment.

The siRNA expressed from a recombinant plasmid may be isolated from a cell expression system cultured by standard technology, or may be intracellularly expressed in or near an angiogenetic area in the living body.

The siRNA of the present invention may be expressed from a recombinant plasmid as two different complementary RNA molecules or a single RNA molecule with two complementary areas.

The selection of a plasmid suitable for expressing the siRNA of the present invention, a method of inserting a nucleic acid sequence to express siRNA in the plasmid, and a method of delivering the recombinant plasmid to desired cells is known to those of ordinary skill in the art. For example, the following documents may be referenced and are hereby incorporated by reference in their entirety [References: Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508].

In addition, the siRNA of the present invention may be intracellularly expressed from a recombinant viral vector in or near an angiogenetic area in the living body. The recombinant viral vector of the present invention includes a sequence encoding siRNA of the present invention, and any suitable promoter for expressing the siRNA sequence. Suitable promoters include, for example, a U6 or H1 RNA pol III promoter sequence and a cytomegalovirus promoter. Other suitable promoters may be selected by those of ordinary skill in the art. In addition, the recombinant plasmid of the present invention may include an inducible or regulatory promoter for expressing siRNA in a specific tissue or intracellular environment.

The siRNA of the present invention may be expressed from a recombinant viral vector as two different complementary RNA molecules or a single RNA molecule with two complementary areas.

Any viral vector capable of carrying the coding sequence for the siRNA molecule to be expressed may also be used, including, for example, vectors derived from an adenovirus (AV), an adenovirus-associated virus (AAV), a retrovirus (e.g., a lentivirus (LV), a rhabdovirus, murine leukemia virus), and a herpes virus. In addition, the tropism of the viral vector may be changed by pseudotyping the vector with an envelope protein or other surface antigens of a different virus. For example, the AAV vector of the present invention may be pseudotyped with a surface protein of vesicular stomatitis virus (VSV), rabies virus, Ebola virus, or Mokola virus.

The selection of a recombinant viral vector suitable to be used in the present invention, a method of inserting a nucleic acid into the vector to express siRNA, and a method of delivering the vector to desired cells is known to those of ordinary skill in the art. For example, the following documents may be referenced, which are hereby incorporated by reference in their entirety [References: Dornburg R (1995), Gene Therap. 2: 301-310; Eglitis MA 1988), Biotechniques 6: 608-614; Miller AD (1990), Hum Gene Therap. 1: 5-14; and Anderson WF (1998), Nature 392: 25-30]. In addition, the siRNA of the present invention is in the form of an oligonucleotide, and may be transfected into somatic cells using a delivery buffer.

Since these genes themselves have a phosphate structure and are negatively charged, it is not easy for the genes themselves to penetrate the cell membrane, which is negatively charged, due to electrical repulsion. Therefore, the gene reacts with a positively charged material to form a complex and the overall charge should be positive to enter cells more easily and enhance the expression of the gene in the cells. Like this, a material for enhancing the delivery of the gene into cells is called a carrier. The carrier means a material that is combined with a gene and helps deliver the gene for enhanced delivery and high expression of the gene, and such a gene carrier forms a gene/carrier complex by electrical interaction between a negatively-charged gene and a positively-charged gene carrier.

According to one embodiment of the present invention, using a non-viral gene carrier (hereinafter, referred to as TKPR-9R) including tuftsin-oligoarginine, inflammatory macrophages may be targeted. The inflammatory macrophage-targeting peptide consists of tuftsin (TKPR peptide sequence) enabling selective targeting of macrophages in an organ (liver, lung, kidney, spleen) and peritoneal fluid and a sequence of 9 arginines (RRRRRRRRR, 9R) whose positive charge ensures introduction into cells.

The TKPR-9R includes cysteines at both ends.

The TKPR-9R refers to ‘Cys-TKPR-(9Arg)-Cys’ peptide [Cys Thr Lys Pro Arg Arg Arg Arg Arg Arg Arg Arg Arg Arg Cys: SEQ ID NO: 5]. Here, the D-form of arginine was used.

First, the ‘Cys-TKPR-(9Arg)-Cys’ peptide may be synthesized using a solid-phase Fmoc peptide synthesis method, which is a synthesis method of elongating a peptide chain by adding each amino acid one by one according to a specified sequence and uses an amino acid whose α-amino group is protected by 9-fluorenylmethoxycarbonyl (Fmoc). After the peptide chain has been elongated, it is treated with trifluoroacetic acid (TFA) to obtain a free peptide.

A therapeutic gene, i.e., siTACE, has a limitation in clinical application due to a short half-life and low cell targeting and permeability. For effective gene treatment, siTACE should pass through the cell membrane to ensure effective silencing of TACE mRNA in the cytoplasm in a target cell. Accordingly, in the present invention, a TKPR-9R gene carrier was used as a macrophage-targeting carrier to enhance the in vivo stability of the therapeutic gene.

The human siTACE and TKPR-9R form a complex through an electrical interaction, and to form a gene/carrier complex (peptoplex) having an excellent therapeutic effect, the optimal ratio of a gene and a gene carrier, which form the complex, is required. Although there are several types of ratios, such as weight ratio, charge ratio, and N/P ratio, the present invention uses weight ratio. The gene/carrier complex according to the present invention is preferably formed of TACE siRNA and TKPR-9R in a weight ratio of 1:1.2 to 5 or 1:3 to 4.5.

In sepsis caused by infection, macrophages in the body overexpress TACE, and thus release TNF-α and triggers an inflammatory response, leading to severe damage to organs. Therefore, when siTACE is specifically delivered to macrophages, it is possible to effectively inhibit the inflammatory response without side effects.

In addition, the present invention includes a method of preparing a gene/carrier complex, which includes mixing and incubating siRNA suppressing TACE expression and a non-viral gene carrier.

The incubation is preferably performed at 20 to 40° C. for 20 to 40 minutes to form an optimal gene/carrier complex. At a high temperature exceeding 40° C., interactions between bases of DNA are dissociated, resulting in denatured DNA. The carrier is formed by peptide polymerization and is also denatured at high temperatures, so the incubation is preferably performed at a temperature of 40° C. or less. To treat cells with a complex, the complex is preferably formed at a temperature similar to the body temperature. Meanwhile, when the incubation time exceeds 40 minutes, a gene and carrier form a precipitate, so it is preferable to not exceed 40 minutes. In addition, after combining the gene and the carrier through electrical attraction, it is preferable to incubate the complex for at least 20 minutes to maintain a stable state of the complex.

siTACE is negatively charged, and a non-viral carrier is positively charged. When these two are combined and incubated at room temperature for 20 to 40 minutes, the gene/carrier complex may be formed through electrostatic attraction. After forming the complex, the final volume is adjusted equally for each group using 3DW, PBS and the like.

To obtain the optimal weight ratio of the gene/carrier complex, first, each concentration of the gene and gene carrier should be calculated. Since an amount of absorbed UV radiation is proportional to the amount of DNA, a gene concentration is measured using an UV spectrophotometer. To prevent the precipitation of the gene/carrier complex, a common gene concentration is preferably 1 mg/mL or less. The gene carrier is synthesized at a final concentration of 1 mg/mL by adjusting the amount of HEPES buffer solution.

Here, all contents described in relation to the gene/carrier complex may be directly used or applied to the method of preparing a gene/carrier complex.

The gene/carrier complex according to the present invention binds to an NRP-1 receptor on the surface of a macrophage, thereby selectively enhancing a macrophage-targeting effect.

In particular, the gene/carrier complex according to the present invention binds to an NRP-1 receptor on the surface of a macrophage, thereby selectively enhancing a macrophage-targeting effect.

In addition, the intravenous injection of the siTACE/TKPR-9R gene/carrier complex into experimental animals, i.e., mice, shows a targeting effect on peritoneal macrophages of the lungs, liver, kidneys, and spleen, as well as macrophages in tissue where inflammation occurs.

When administered in combination with an antibiotic currently used for acute inflammatory diseases (e.g., sepsis), it was confirmed that the inflammatory signaling system in each organ was alleviated and the gene therapy resulted in an improved survival rate.

Accordingly, the present invention provides a composition for preventing or treating an acute inflammatory disease, which includes the gene/carrier complex as an active ingredient.

The acute inflammatory disease may be acute sepsis, acute lung injury, acute liver failure, or acute inflammatory bowel disease, but the present invention is not limited thereto.

The acute lung injury may include acute pneumonia caused by viruses, bacteria, etc., the acute liver failure may include acute hepatitis caused by viruses, bacteria, etc., and the acute inflammatory bowel disease may include infectious enteritis.

The pharmaceutical composition of the present invention may be administered in combination with a pharmaceutically acceptable carrier, and for oral administration, may further include, other than the active ingredient, a binder, a lubricant, a disintegrant, an excipient, a solubilizer, a dispersant, a stabilizer, a suspending agent, a coloring agent, and a flavor. For an injection, the pharmaceutical composition of the present invention may be used by mixing a buffer, a preservative, a pain relief agent, a solubilizer, an isotonic agent and a stabilizer. In addition, for topical administration, the composition of the present invention may use a base material, an excipient, a lubricant, and a preservative.

The pharmaceutical composition of the present invention may be prepared in various forms by being mixed with the above-described pharmaceutically acceptable carrier. For example, for oral administration, the pharmaceutical composition of the present invention may be prepared in various dosage forms such as a tablet, a troche, a capsule, an elixir, a suspension, a syrup and a wafer, and for injections, the pharmaceutical composition of the present invention may be prepared in a unit dose ampoule or multiple dose forms. The pharmaceutical composition of the present invention may be formulated as a solution, a suspension, a tablet, a capsule or a sustained-release preparation.

Meanwhile, examples of carriers, excipients and diluents suitable for preparation may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. The examples of carriers, excipients and diluents may also include a filler, an anti-agglomerate, a glidant, a wetting agent, a fragrance, and a preservative.

The pharmaceutical composition of the present invention can be orally or parenterally administered. The pharmaceutical composition according to the present invention may be administered, for example, orally, by an aerosol, buccally, via the skin, intradermally, by inhalation, intramuscularly, intranasally, intraocularly, intrapulmonarily, intravenously, intraperitoneally, into ears, by injection, by a patch, subcutaneously, sublingually, topically, or transdermally, but the present invention is not limited thereto.

For such clinical administration, the pharmaceutical composition of the present invention may be prepared in a suitable form using known techniques. For example, for oral administration, it may be administered by mixing with an inactive diluent or an edible carrier, by being sealed in a hard or soft gelatin capsule, or compressed into a tablet. In the pharmaceutical composition for oral administration, the active ingredient may be mixed with an excipient to be used in the form of an ingestible tablet, a buccal tablet, a troche, a capsule, an elixir, a suspension, a syrup, or a wafer. In addition, various dosage forms for injection and parenteral administration may be prepared according to a technique that is known or commonly used in the art.

An effective dose of the pharmaceutical composition of the present invention may vary according to a patient's weight, age and sex, a health condition, a diet, administration time, an administration method, an excretion rate and the severity of a disease, and may be easily determined by those of ordinary skill in the art.

A preferred dose of the pharmaceutical composition of the present invention may vary according to a patient's condition and weight, the severity of a disease, a form of the drug, an administration route, and duration, but may be suitably selected by those of ordinary skill in the art. However, the pharmaceutical composition of the present invention is preferably administered daily at 0.001 to 100 mg/kg of body weight, more preferably, at 0.01 to 30 mg/kg of body weight. The daily dose of the pharmaceutical composition of the present invention may be administered once a day or in divided portions. The gene carrier complex of the present invention may be present at 0.0001 to 10 wt %, and preferably, 0.001 to 1 wt % with respect to the total weight of the entire composition.

The pharmaceutical composition of the present invention may be administered to a mammal such as a rat, a mouse, stock, or a human via various routes. There is no limit on the administration method, and for example, the pharmaceutical composition of the present invention may be administered orally, or by intrarectal, intravenous, intramuscular, or subcutaneous, intrauterine dural, or intracerebroventricular injection.

Accordingly, the present invention provides a method of preventing or treating an acute inflammatory disease, which includes administering a therapeutically effective amount of pharmaceutical composition containing the complex to a subject.

The prevention or treatment method of the present invention includes administering a therapeutically effective amount of the composition of the present invention. The therapeutically effective amount means an amount that enhances an acute inflammation inhibitory effect. It is obvious to those of ordinary skill in the art that an appropriate daily dose may be determined by a physician within the scope of sound medical judgement. A specific therapeutically effective amount for a particular patient may depend on the type and degree of response to be achieved, a particular composition, including whether a different agent is used depending on the circumstances, a patient's age, weight, overall health condition, sex, and diet, administration time, an administration route, an excretion rate of the composition, the duration of treatment, and various factors and similar factors well known in the medical field. Therefore, the effective amount of the pharmaceutical composition suitable for the purpose of the present invention is preferably determined by considering the above-mentioned details. In addition, in some cases, the therapeutic effect on a related disease may be enhanced by co-administering the composition of the present invention and a known therapeutic agent for the related disease.

The term “subject” used herein includes mammals such as a horse, sheep, a pig, a goat, a camel, an antelope, and a dog, having a related disease whose symptoms can be alleviated by administering the pharmaceutical composition according to the present invention.

Hereinafter, the present invention will be described in detail with reference to examples. The following examples are merely illustrative of the present invention and the scope of the present invention is not limited to the following examples. These examples are provided to complete the disclosure of the present invention and fully convey the scope of the present invention to those of ordinary skill in the art, and the present invention should be defined by only the accompanying claims.

### EXAMPLES

### Preparation Example 1: Preparation of Mouse siTACE Vector

As a therapeutic gene (siTACE) of a gene silencing system targeting an RNA molecule, based on the mouse TACE (NM_001277266) sequence, sequences each consisting of a total of 19 bases, i.e., 5′-ACACCTGCTGCAATAGTGA-3′(Sense sequence; SEQ ID NO: 1), and 5′-TCACTATTGCAGCAGGTGTT-3′ (Antisense sequence; SEQ ID NO: 3) were produced, and an overhang sequence TT was added at the end of the 3′ end to effectively suppress gene expression. This was achieved by an oligonucleotide chemical synthesis process of siTACE. The synthesis was performed by sequentially adding adenosine, guanosine, cytidine, and uracil, which are basic base materials constituting a gene, using a solid silica support. Each time when one base was added sequentially, an oligonucleotide was synthesized through four steps, including a de-blocking reaction, a coupling reaction, a capping reaction, and an oxidation reaction. Afterward, the resulting oligonucleotide was reacted with ammonia water to recover an oligonucleotide, the synthesized oligonucleotide was purified using the principle of chromatography using a reverse-phase silica resin. Subsequently, a quantitative process was conducted to measure absorbance using an UV spectrophotometer, thereby calculating the number of moles of the synthesized oligonucleotide.

Meanwhile, the therapeutic effect of the gene therapeutic agent system of the present invention was verified by an animal experiment using mice.

### Preparation Example 2: Preparation of Human siTACE Vector

As a therapeutic gene (siTACE) of a gene silencing system targeting an RNA molecule, based on the human TACE (NM_003183.5) sequence, sequences each consisting of a total of 21 bases, i.e., 5′-GCTCTCAGACTACGATATTCT-3′(Sense sequence; SEQ ID NO: 2), and 5′-AGAATATCGTAGTCTGAGAGC-3′ (Antisense sequence; SEQ ID NO: 4) were produced, and an overhang sequence TT was added at the end of the 3′ end to effectively suppress gene expression. This was achieved by an oligonucleotide chemical synthesis process of siTACE. The synthesis was performed by sequentially adding adenosine, guanosine, cytidine, and uracil, which are basic base materials constituting a gene, using a solid silica support. Each time when one base was added sequentially, an oligonucleotide was synthesized through four steps, including a de-blocking reaction, a coupling reaction, a capping reaction, and an oxidation reaction. Afterward, the resulting oligonucleotide was reacted with ammonia water to recover an oligonucleotide, the synthesized oligonucleotide was purified according to the principle of chromatography using a reverse-phase silica resin. Subsequently, a quantitative process was conducted to measure absorbance using an UV spectrophotometer, thereby calculating the number of moles of the synthesized oligonucleotide. To apply this system clinically, an experiment on a human cell line should be conducted first. The anti-inflammatory effect of the gene therapeutic agent in human-derived cells (THP-1) should be verified, and in this case, the human siTACE gene should be used instead of the mouse siTACE gene.

### Preparation Example 3: Preparation of Gene Carrier (TKPR-9R)

An inflammatory macrophage-targeting peptide consists of a TKPR peptide sequence (TKPR) enabling selective targeting of macrophages in organs (liver, lungs, kidneys, and spleen) and peritoneal fluid, and a 9-arginine sequence (RRRRRRRRR, 9R), which facilitates intracellular entry with a positive charge. Here, the D-form of arginine was used.

First, a ‘Cys-TKPR-(9Arg)-Cys’ monomer peptide was synthesized using a solid Fmoc peptide synthesis method. This is a synthesis method that elongates a peptide chain by adding each amino acid one by one according to a specified sequence order and uses an amino acid whose α-amino group is protected by an Fmoc group. After elongation of the peptide chain, it was treated with TFA to obtain a free peptide. Afterward, the TFA salt-type ‘Cys-TKPR-(9Arg)-Cys’ monomer peptide was replaced with an acetic acid salt type. The salt form was replaced with an acetic acid salt through ion exchange chromatography using AG1-X8 resin.

‘Cys-TKPR-(9Arg)-Cys’=Cys Thr Lys Pro Arg Arg Arg Arg Arg Arg Arg Arg Arg Arg Cys [SEQ ID NO: 5]

### Preparation Example 4: Preparation of siTACE/TKPR-9R Complex

(The method of preparing an siTACE/TKPR-9R complex was performed using human siTACE instead of mouse siTACE.)

siRNA suppressing TACE expression and a non-viral gene carrier (TKPR-9R) were prepared at 1 mg/mL. The gene and peptide were added to a PBS solution with a volume 4-fold the total volume of the gene and carrier, and incubated at room temperature (25° C.) for 30 minutes. When the incubation time exceeded 40 minutes, the gene and carrier formed a precipitate, so the incubation time is preferably less than 40 minutes.

### Example: Formation of Gene/Gene Carrier Complex and Verification of its Efficacy

[Experimental Process]

<Materials>

Fetal bovine serum (FBS) and Dulbecco's Modified Eagle's Medium (DMEM) containing high glucose were purchased from WELGENE (Seoul, Korea).

Lipopolysaccharide (LPS), lactic acid (LA), polyethyleneimine (PEI, branched 25 kDa form), and 3-[4,5-dimethylthiazol-2-yl]-2,5-diphenyl tetrazolium bromide (MTT) solution were purchased from Sigma-Aldrich (St. Louis, USA).

Antibodies (anti-TACE and anti-GAPDH) were purchased from Abcam (Cambridge, MA, USA).

A poly(vinylidene fluoride) (PVDF) transfer membrane was purchased from Millipore Sigma (Burlington, MA, USA).

Mouse ELISA kits (TNF-α, IL-1β, and MCP-1) were purchased from ThermoFisher Scientific (USA), and a mouse ELISA kit (IL-6) was purchased from eBioscience (USA).

<Agarose Gel Electrophoresis>

A gene therapeutic agent complex was formed by incubating 1 μg of siTACE therapeutic gene (siRNA) with various amounts (0.5, 1, 2, 3, and 4 μg) of TKPR-9R carriers at room temperature for 30 minutes. Afterward, complex formation was confirmed according to charge comparison through electrophoresis on a 0.8% (w/v) agarose gel in a 0.5×TBE buffer solution at 100 V for 20 minutes.

<Measurement of Surface Charge and Size of Complex>

A complex was formed by incubating 5 μg of siTACE therapeutic gene with the siTACE/TKPR-9R complex at various weight ratios (weight ratios of TKPR-9R/siTACE=1, 2, 3, and 4) at room temperature for 30 minutes, and then the total volume was adjusted to 800 μL with deionized water. The surface charge and size of the complex was measured using a Zeta sizer-ZS (Malvern) machine.

<Analysis of Binding Ability of TKPR Peptide to Raw263.7 Macrophages>

After differentiation into M1 type inflammatory macrophages by LPS, for an experiment for analyzing TKPR peptide-induced competitive binding ability, a free-TKPR-9R peptide was first treated at room temperature for 2 hours. Afterward, a FAM fluorescence-conjugated siRNA gene (FAM-siRNA) and the TKPR-9R peptide were reacted at room temperature for 30 minutes, the degree of introduction of the complex into macrophages was analyzed through FACS. In addition, in a group (Naked-siRNA-FAM) in which macrophages were treated with only the siRNA-FAM gene without the TKPR-9R peptide, the degree of gene introduction into macrophages was analyzed through FACS.

<Cell Culture>

DMEM and FBS were purchased from WELGENE (Korea) to form a medium. Raw 264.7 mouse-derived macrophages were purchased from the Korean Cell Line Bank, and subcultured in a fresh medium every other day. The cells were incubated in a complete medium supplemented with 10% FBS, penicillin (100 IU/mL) and streptomycin (100 μg/mL) in an incubator at 37° C. under a 5% CO2 atmosphere.

<Measurement of TACE mRNA (Isolation of Intracellular RNA and Real-Time PCR)>

Mouse-derived macrophages (Raw 264.7 macrophages) were seeded at 4×104 cells/well in a 12-well cell culture plate and incubated for 24 hours. After treating the macrophages with 1 μg/mL of LPS for 1 hour, an siTACE/TKPR-9R gene/carrier complex was treated for 24 hours. Afterward, the cells were homogenized using aa RNeasy Mini kit (Qiagen), and only RNA was isolated. The isolated RNA was reacted with reverse transcriptase using an iScript cDNA synthesis kit (Bio-Rad) to synthesize cDNA complementary to 1 μg of RNA for each group. Subsequently, an amount of TACE mRNA relative to the endogenous control GAPDH was calculated by real-time PCR using a Cyber premix Ex taq RT-PCR kit. (In the case of a mouse, for TACE, the forward primer was 5′-GTACGTCGATGCAGAGCAAA-3′[SEQ ID NO: 6] and the reverse primer was 5′-AAACCAGAACAGACCCAACG-3′ [SEQ ID NO: 7]; and in the case of a human, for TACE, the forward primer was 5′-TGAAGAGCTTGTTCATCGAG-3′ [SEQ ID NO: 8], and the reverse primer was 5′-CCATGAAGTGTTCCGATAGATGTC-3′[SEQ ID NO: 9].)

<Measurement of Water-Soluble (Inflammatory) TNF-α (ELISA)>

Mouse-derived macrophages (Raw 264.7 macrophages) were incubated at 4×104 cells per well in a cell culture plate for 24 hours. Afterward, except a lactic acid (LA)-treated group as a positive control, the cells were treated with 1 μg/mL/well of LPS for 1 hour to induce an inflammatory state of the macrophages. The LA-treated group was intended to activate M2-type macrophages. The inflammatory macrophages were treated with the siTACE/TKPR-9R gene/carrier complex for 48 hours. Afterward, the culture medium was obtained from each well and centrifuged at 4° C. and 13000 rpm for 5 min, thereby obtaining a supernatant of the medium as a sample. The amount of a water-soluble TNF-α inflammatory mediator (cytokine) was measured from the medium sample through sandwich ELISA.

<Septic Animal Modeling and Injection of Gene/Carrier Gene Therapeutic Agent>

C57BL/6 experimental animals was used for sepsis models induced by a surgical method of cecal ligation and puncture (CLP). After 24 hours, a gene/carrier complex (siTACE/TKPR-9R complex) formed of 20 μg of siTACE therapeutic gene and 80 μg of TKPR-9R carrier at the optimal weight ratio of 1:4 was injected into the tail vein. Here, for the CLP method, anesthesia was performed with 2,2,2-tribromoethanol (250 mg/kg), the end of the cecum in the abdomen was ligated 1 to 1.5 cm with 4-0 black silk suture, 2 punctures were made with a syringe needle, the feces in the cecum were guided out of the punctures by pressing the cecum with surgical forceps, and the peritoneum was stitched with 4-0 black silk suture. The dose of gene therapeutic agent administered to the experimental animal was approximately 1 mg/kg.

<Measurement of Survival Rate>

After administering the gene therapeutic agent to the sepsis model induced by the surgical method of CLP, gentamicin (8 mg/kg, I.P.) and cephalosporin (8 mg/kg, I.P.), which are antibiotics, were co-administered, and changes in survival rate caused by the effect of co-administration were confirmed for five days.

<Measurement of Binding Ability to Macrophages in Peritoneal Fluid>

Experimental animals, mice, were dissected and intraperitoneally injected with PBS, thereby obtaining macrophages in peritoneal fluid. To isolate only macrophages, the peritoneal fluid was centrifuged at 300 g for 3 minutes, primary macrophages were incubated in a 12-well plate at 37° C. under a 5% CO2 atmosphere for 24 hours. Afterward, the macrophages were treated with a gene/carrier complex (FAM-siRNA/TKPR-9R complex) for 4 hours, washed with PBS, fixed with 4% paraformaldehyde (PFA) for 30 minutes, and the degree of introduction of a gene therapeutic agent into macrophages was analyzed using macrophage-targeting anti-F4/80 antibodies by FACS Calibur (BD Biosciences).

<Ex Vivo Sampling>

After completing the treatment of a sepsis model induced by the surgical method of CLP with a gene therapeutic agent, the experimental animal was dissected to harvest organs (liver, lungs, kidneys, and spleen) and serum. Organ tissue samples were physically ground, treated with a reporter lysis 5× buffer (Promega) and 0.1 mM PMSF (protease inhibitor), and centrifuged at 16,800 rpm and 4° C. for 30 minutes, thereby obtaining a protein-containing supernatant. The protein sample was boiled with Laemmli buffer for 10 minutes, subjected to 10% SDS-PAGE gel electrophoresis, and a TACE protein expression level was measured using a polyvinylidene fluoride membrane (Millipore), a trans-blot turbo transfer system (Bio-Rad), and anti-TACE antibodies. In addition, some organ samples were homogenized in RLT buffer (Qiagen), RNA was obtained, cDNA was synthesized using an iScript cDNA synthesis kit, and an amount of TNF-α mRNA relative to the endogenous control GAPDH was measured by real-time PCR. (In the case of a mouse, the forward primer for TNF-α was 5′-TCTCATGCACCACCATCAAGGACT-3′ [SEQ ID NO: 10], and the reverse primer for TNF-α was 5′-ACCACTCTCCCTTTGCAGAACTCA-3′ [SEQ ID NO: 11]; and in the case of a human, the forward primer for TNF-α was 5′-CTCTTCTGCCTGCTGCACTTTG-3′ [SEQ ID NO: 12], and the reverse primer for TNF-α was 5′-ATGGGCTACAGGCTTGTCACTC-3′ [SEQ ID NO: 13].) Finally, after blood collection, the blood sample was incubated for 30 minutes at room temperature to form blood clots, centrifuged at 1500 g and 4° C. for 10 minutes, and serum was isolated and inflammatory cytokine and chemokine mediators such as TNF-α, IL-1β, IL-6, MCP-1 in the blood were analyzed by ELISA.

<Cell Culture>

RPMI (Dulbecco's Modified Eagle Medium) and fetal bovine serum (FBS) were purchased from WELGENE (Korea) to form a medium. THP-1 human-derived macrophages were purchased from the Korea Cell Line Bank and subcultured every other day in a fresh medium. The macrophages were incubated in a complete medium supplemented with 10% FBS, penicillin (100 IU/mL), and streptomycin (100 μg/mL) at 37° C. in an incubator under a 5% CO2 atmosphere.

<Analysis of Binding Ability of TKPR Peptide to THP-1 (Macrophage)>

After differentiation into M1-type inflammatory macrophages by lipopolysaccharide (LPS) to conduct an assay for a competitive binding ability induced by a TKPR peptide, a free-TKPR-9R peptide was first treated at room temperature for 2 hours. Afterward, a FAM fluorescence-conjugated siRNA gene (FAM-siRNA) and the TKPR-9R peptide were reacted at room temperature for 30 minutes, and the degree of introduction of the complex into the macrophages was analyzed by FACS. In addition, in the group in which macrophages were treated with only the siRNA-FAM gene without the TKPR-9R peptide (Naked-siRNA-FAM), the degree of gene-induced introduction into macrophages was analyzed by FACS.

<TACE mRNA Measurement (Isolation of RNA in Cells and Teal-Time PCR)>

Mouse-derived macrophages (THP-1 macrophages) were seeded at 4×104 cells/well in a 12-well plate and incubated for 24 hours. After treating the macrophages with 1 μg/mL of LPS for 1 hour, an siTACE/TKPR-9R gene/carrier complex was treated for 24 hours. Afterward, the cells were homogenized using a RNeasy Mini kit (Qiagen), and only RNA was isolated. The isolated RNA was reacted with reverse transcriptase using an iScript cDNA synthesis kit (Bio-Rad) to synthesize cDNA complementary to 1 μg of RNA for each group. Amounts of TACE, TNF-α, and IL-1beta mRNA relative to the endogenous control GAPDH were measured through real-time PCR using a Cyber premix Ex taq RT-PCR kit. (In the case of a mouse, for TACE, the forward primer was 5′-GTACGTCGATGCAGAGCAAA-3′[SEQ ID NO: 6], the reverse primer was 5′-AAACCAGAACAGACCCAACG-3′ [SEQ ID NO: 7]; and in the case of a human, for TACE, the forward primer was 5′-TGAAGAGCTTGTTCATCGAG-3′ [SEQ ID NO: 8], and the reverse primer was 5′-CCATGAAGTGTTCCGATAGATGTC-3′ [SEQ ID NO: 9].) (In the case of a mouse, for TNF-α, the forward primer was 5′-TCTCATGCACCACCATCAAGGACT-3′ [SEQ ID NO: 10], and the reverse primer was 5′-ACCACTCTCCCTTTGCAGAACTCA-3′ [SEQ ID NO: 11]; and in the case of a human, for TNF-α, the forward primer was 5′-CTCTTCTGCCTGCTGCACTTTG-3′ [SEQ ID NO: 12], and the reverse primer was 5′-ATGGGCTACAGGCTTGTCACTC-3′ [SEQ ID NO: 13].) (In the case of a mouse, for IL-1beta, the forward primer was 5′-TGGACCTTCCAGGATGAGGACA-3′ [SEQ ID NO: 14], and the reverse primer was 5′-GTTCATCTCGGAGCCTGTAGTG-3′ [SEQ ID NO: 15], and in the case of a human, for IL-1beta, the forward primer was 5′-GGACAAGCTGAGGAAGATGC-3′ [SEQ ID NO: 16], and the reverse primer was 5′-TCCATATCCTGTCCCTGGAG-3′ [SEQ ID NO: 17].)

[Experimental Results]

1. Physicochemical Characteristics and In Vitro Macrophage Targeting Ability of siTACE/TKPR-9R Complex

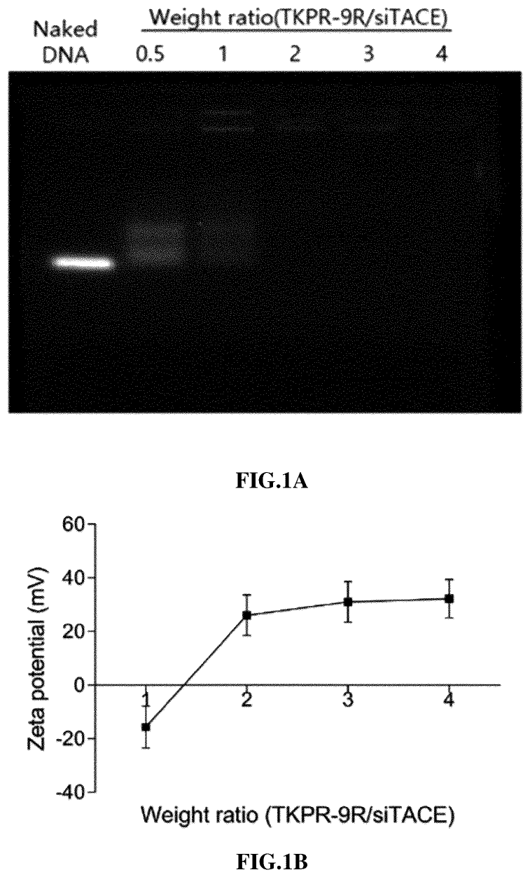

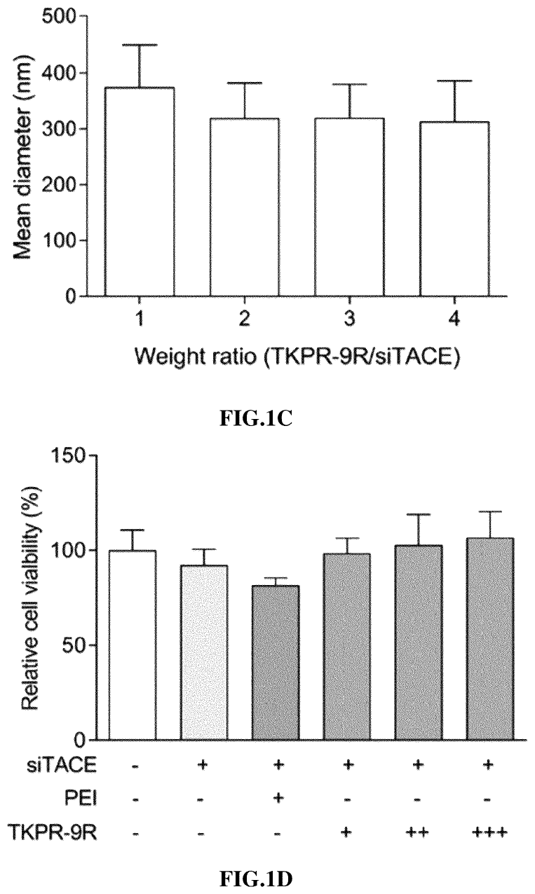

For effective gene therapy, a therapeutic gene should be delivered. The gene is expressed in target cells. However, both therapeutic siRNA and the cell membrane have negative charges, so it is difficult to introduce the gene into macrophages. Accordingly, characterization experiments were conducted to optimize the weight ratio of the siTACE/TKPR-9R complex. After incubating siTACE and TKPR-9R in deionized water for 30 minutes, an agarose gel retardation test was conducted to determine whether the siTACE/TKPR-9R complex was condensed and stabilized (FIG. 1A). Stable formation of the siTACE/TKPR-9R complex was confirmed at a ratio of 1 or more. On the other hand, siRNA of a group with a weight ratio of less than 1 moved down from the top of the agarose gel due to ionic repulsion. At the siTACE/TKPR-9R weight ratio of 4, the siTACE/TKPR-9R complex showed a positive charge (surface charge with a zeta potential of 32.2(±7.2) mV, a nano complex size of 313.7 nm (±72.45), and a polydispersity index (PDI) value of 0.208 (FIGS. 1B and 1C).

After 24-hour incubation of mouse siTACE/TKPR-9R (optimal weight ratio of TKPR-9R/siRNA=4), the mean diameter and surface zeta potential were maintained, and sizes were well distributed with a PDI value of 0.158 at the optimum weight ratio of 4. In addition, the optimal weight ratio of 4 showed a non-toxic effect on the Raw264.7 cells, indicating that the TKPR-9R peptide is safer than PEI (FIG. 1D). The cellular uptake of the siRNA-FAM/TKPR-9R complex (siRNA: TKPR-9R weight ratio=1:4) was higher than that of the complex at a weight ratio of 1:3. In addition, a competition assay using a free TKPR peptide was conducted to verify the macrophage-specific targeting ability of TKPR-9R (FIG. 1E). Raw264.7 was activated by LPS for 2 hours. After activation, a free TKPR peptide was incubated for 1 hour to block receptors on macrophages. siRNA alone did not show a change in fluorescence intensity when an NRP-1 receptor was blocked. However, the mean fluorescence intensity (MFI) of the complex significantly decreased when the receptor was occupied by free TKPR. As a result, the cellular uptake of siRNA was mediated by TKPR directly binding to the receptor of the macrophage.

Consequently, activated macrophages having NRP-1 receptors in inflammatory tissue may be specifically targeted through the TKPR sequence of the peptide. In addition, the siTACE/TKPR-9R complex was efficiently internalized into the cytoplasm of the Raw 264.7 cells.

(The optimal weight ratio of the TKPR-9R/siRNA complex shown in the subsequent experiment was used as a weight ratio of 4/1 (gene:carrier=1:4).)

2. In Vitro TACE Gene Silencing Effect of siTACE/TKPR-9R Complex in Mouse Macrophage Raw 264.7 Cell Line

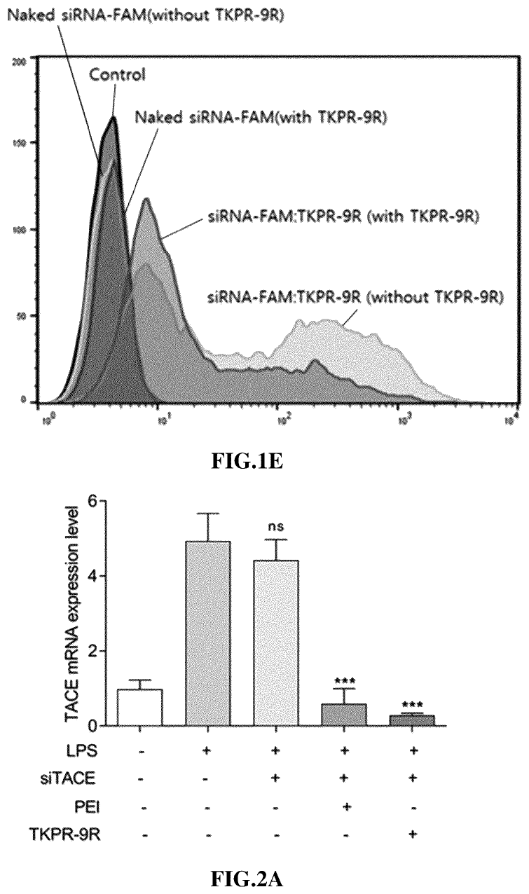

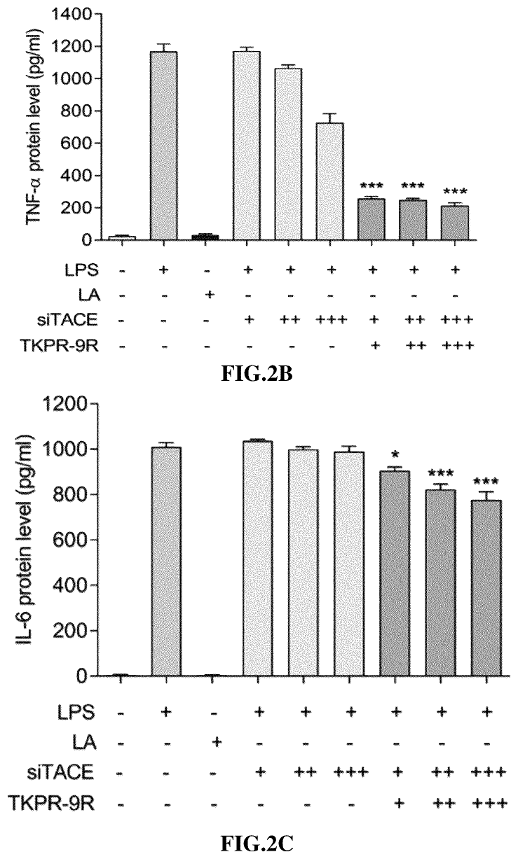

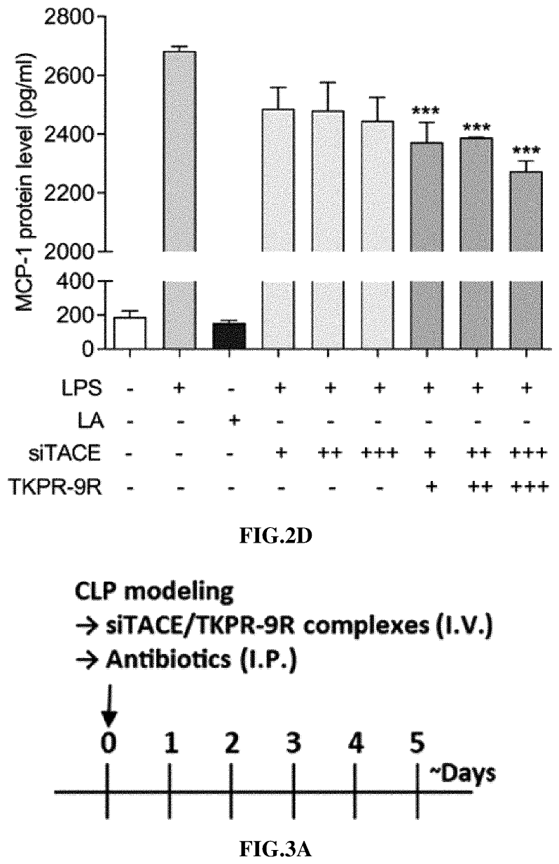

TKPR-mediated delivery of siTACE showed inhibition of TACE in inflammatory macrophages. Transcription of the TACE gene was significantly inhibited when using a cationic polymer or peptide (FIG. 2A). However, PEI showed high cytotoxicity, and PEI was off-target. The effect may lead to serious failures in sepsis treatment. In contrast, TKPR-9R showed better transcriptional inhibition of TACE with a high survival rate and no toxicity in Raw 264.7 macrophages (FIG. 2D). TNF-α expression was also reduced in a dose-dependent manner (FIG. 2B). To compare the TNF-α expression level between pro-inflammatory and anti-inflammatory macrophages, LA was used for M2 type-macrophage polarization. A high dose of siTACE/TKPR-9R complex up-regulated the degradation of TNF-α protein, resulting in a similar protein level to that of the LA-treated group, which differentiated monocytes into M2 phenotype macrophages. Even siRNA having no TKPR-9R carrier showed the inhibition of TNF-α in a dose-dependent manner but was not as effective as the siTACE/TKPR-9R complex.

In addition, the quantitative protein level analysis of cytokines and chemokines showed down-regulated levels in the group treated with the siTACE/TKPR-9R complex (FIGS. 2C and 2D). LPS-activated macrophages to excrete a variety of pro-inflammatory cytokines and chemokines, such as IL-6 and MCP-1, whereas LA attenuated the cytokine and chemokine levels.

Cytokine release from LPS-activated macrophages was inhibited in a dose-dependent manner by treatment with the siTACE/TKPR-9R complex. siTACE alone showed weak interfering effects on TACE mRNA and inflammatory cytokines, but the siTACE/TKPR-9R complex dramatically increased its efficacy. Therefore, TKPR-9R can be expected to increase a gene silencing effect in in vivo experiments compared to siRNA alone. In addition, this anti-inflammatory effect was further increased when the siTACE gene was combined with the TKPR-9R carrier, but it was insufficient when the gene or carrier as used alone.

3. Combination Therapy with siTACE/TKPR-9R Complex and Antibiotic in Sepsis Mouse

The therapeutic effect of the siTACE/TKPR-9R complex was investigated in a severe septic mouse model by CLP surgery. This CLP modeling method is the most pathologically similar to a human clinical disease (polymicrobial sepsis) because it contaminates the peritoneal cavity with feces and bacteria from the appendix.

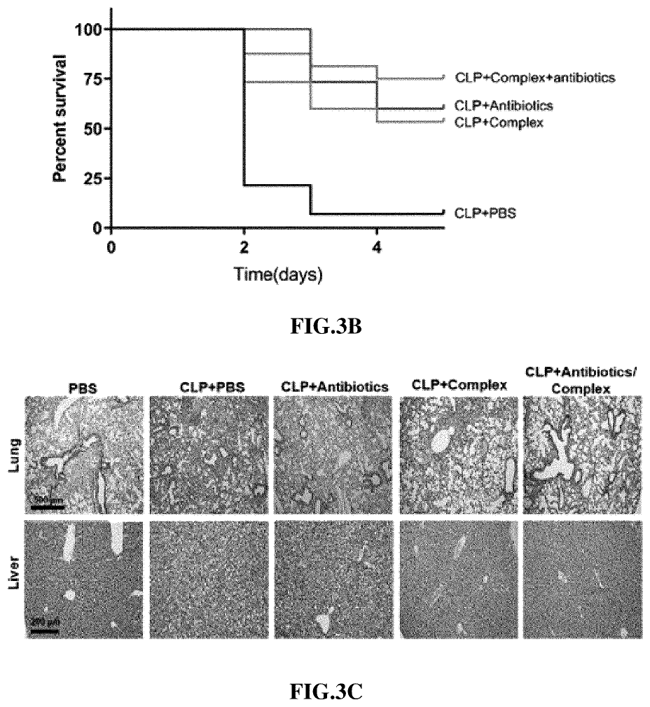

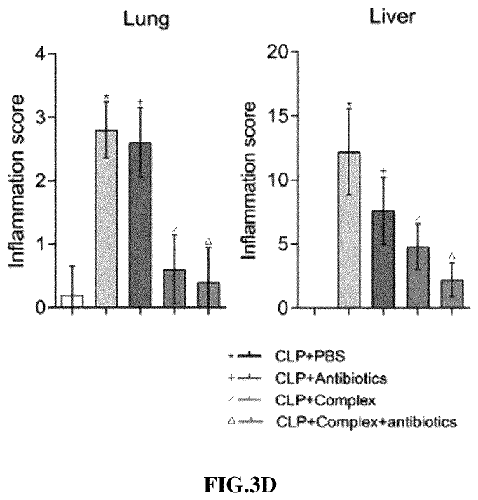

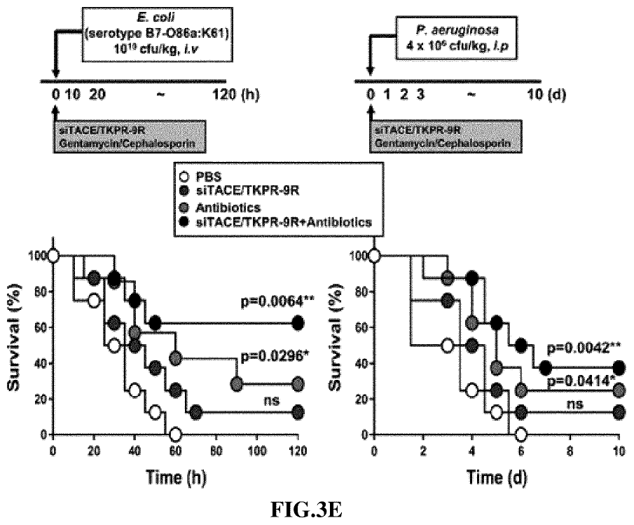

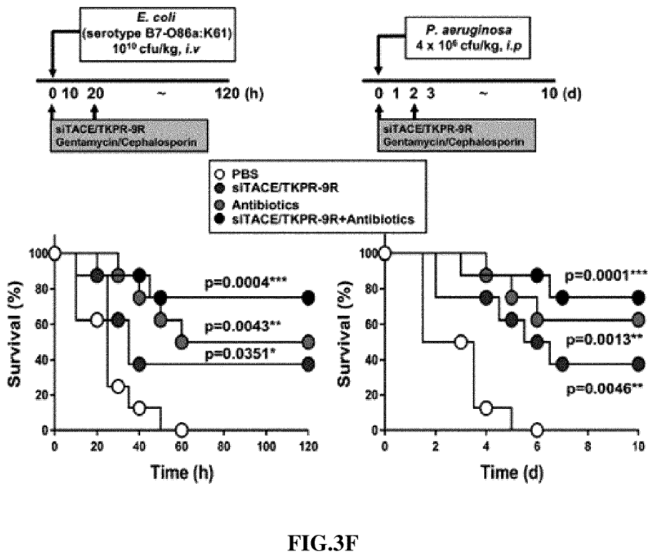

After intravenously injecting the siTACE/TKPR-9R complex (siTACE gene: 1 mg/kg) into a septic mouse, the protective effect was examined by monitoring the survival rate for 5 days in a CLP-induced sepsis model (FIG. 3A). While the PBS-injected control showed a 7% survival rate, the siTACE/TKPR-9R-injected group showed a significant improvement to 53% by attenuating a fatal inflammatory cascade (FIG. 3B). The difference in survival rate between the antibiotic-administered group and the siTACE/TKPR-9R-administered group was merely 7%. The synergistic effect of combination treatment of an antibiotic and the siTACE/TKPR-9R complex was confirmed with a high survival rate of 73%. Consistent with the survival rate data, the clinical improvement of the siTACE/TKPR-9R complex was proven by histological analysis in lung and liver tissue sections of the CLP-induced septic mouse (FIGS. 3C and 3D). Each organ was harvested, and the sections were stained with H&E. Pathologists scored histological scores, and the severity of sepsis was remarkably lower in the siTACE/TKPR-9R group than that in the PBS group.

In addition, the protective effect of the siTACE/TKPR-9R (siTACE gene: 1 mg/kg) complex was proven in a bacteria-induced septic mouse which had been injected with E. coli and P. aeruginosa (FIG. 3E). By the combination administration of the siTACE/TKPR-9R complex and an antibiotic, the survival rate significantly increased compared to the administration of an antibiotic alone. In addition, the survival rate of two injections was higher in the group to which siTACE/TKPR-9R (siTACE gene: 1 mg/kg) and antibiotics (gentamycin: 8 mg/kg, cephalosporin: 8 mg/kg) were administered at the same time. Compared to that of single injection, the double injections took a longer time (FIGS. 3E and 3F).

4. Anti-Inflammatory Effect of siTACE/TKPR-9R Complex Against Severe Inflammation in Sepsis

To confirm the ant-inflammatory effect of the siTACE/TKPR-9R complex by breaking the positive feedback chain reaction of TNF-α, a TACE level was measured in tissue. Tissue was obtained from each mouse 24 hours after the complex was injected into the CLP-induced mouse.

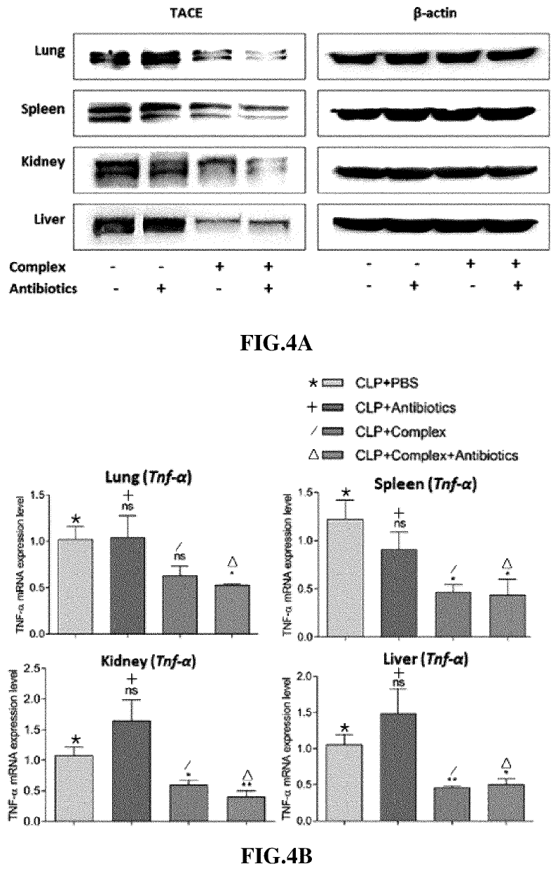

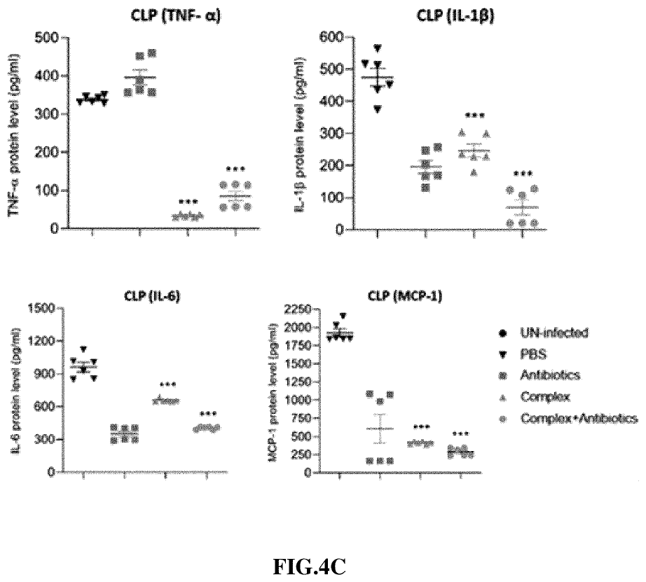

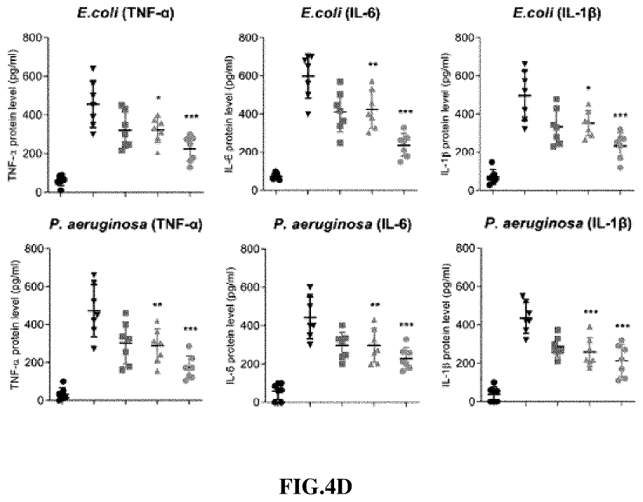

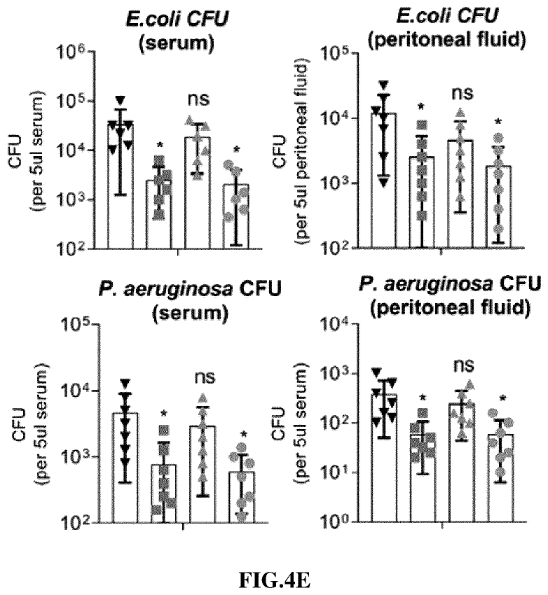

The protein level of TACE was significantly reduced in the siTACE/TKPR-9R complex-administered group, but not in the antibiotics-only group (FIG. 4A). In addition, in the group administered both the complex and the antibiotics, a more significant decrease in TACE level was shown. In addition, the complex affects the transcription of the Tnf-α gene in the lungs, kidneys, spleen and liver (FIG. 4B). Since antibiotics alone did not reduce a TNF-α mRNA level, soluble TNF-α protein itself seemed to be the key molecule involved in promotion. The reduced TACE protein in the complex-treated group interfered with the positive feedback loop of inflammation.

Accordingly, the siTACE/TKPR-9R complex suppressed a circulating cytokine level in mice (FIG. 4C). The decrease in soluble TNF-α may ease the activation of NF-κB that promotes IL-6 secretion. This is because TNF-α is an early cytokine in inflammatory stimulation to induce the release of other pro-inflammatory cytokines such as IL-6.

This group showed a significant decrease in TNF-α level in serum compared to the control group and the antibiotic-treated group. However, IL-1β and IL-6 were reduced in the antibiotic-treated group, indicating that antibiotics have a synergistic effect with the complex to treat septic inflammation. The chemokine (MCP-1) level was reduced in the complex-treated group, which may explain that the down-regulation of TNF-α reduces the infiltration of inflammatory macrophages in the lungs and liver (FIGS. 3C and 4C). In addition, the spleen of the septic mouse was enlarged due to severe inflammation in the PBS- or antibiotic-treated group, but the size of the spleen was reduced in the siTACE/TKPR-9R-treated group.

Such findings show that siTACE/TKPR-9R successfully hinders TACE in tissue macrophages and inhibited the expression of inflammation-related cytokines and chemokines and TNF-α, which can damage tissue.

Moreover, the group treated with the siTACE/TKPR-9R complex and antibiotics showed an anti-inflammatory effect that down-regulates pro-inflammatory cytokine (TNF-α, IL-6, and IL-1β) protein levels in serum of septic mice into which E. coli and P. aeruginosa were injected (FIG. 4D).

In addition, the co-administered group (siTACE/TKPR-9R and antibiotics) showed an antibacterial effect in bacteria-induced septic mice (FIG. 4E). According to this result, the antibiotics and the complex play different roles in anti-inflammatory effects.

5. Macrophage-Targeting Effect of siRNA/TKPR-9R Complex

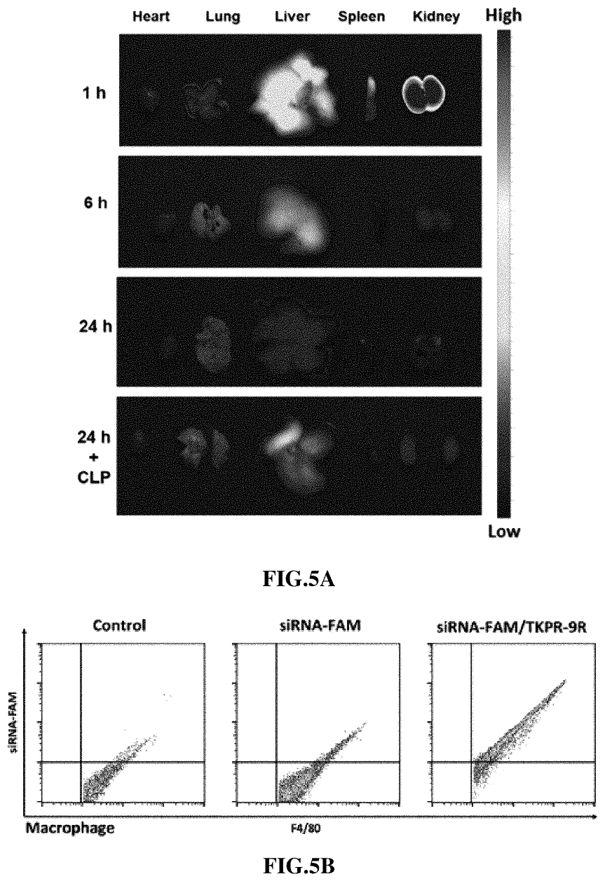

To confirm target gene delivery to macrophages of organs, a Cy5-conjugated-siRNA/TKPR-9R complex was intravenously injected into normal and CLP-induced septic mice (FIG. 5A).

Ex vivo biodistribution images showed the total fluorescence intensity accumulated in the liver, lungs, kidneys and spleen 1 hour after injection. While Cy5 fluorescence was mostly eliminated in the spleen and kidneys of the normal mouse after 24 hours, the accumulation in the septic mice showed higher localization in the liver, spleen, and kidneys. This indicates that the recruitment of tissue-resident macrophages increases during fatal inflammation. However, nano-sized complexes have targeted and non-targeted mechanism in liver accumulation. Accordingly, macrophage internalization with the siRNA/TKPR-9R complex was investigated in normal mice (FIGS. 5B and 5C).

In the peritoneal cavity, the siRNAFAM/TKPR-9R complex was significantly internalized by F4/80 positive primary macrophages. Compared to a naked siRNA-FAM group, the fluorescence intensity of the siRNA-FAM/TKPR-9R complex showed a 9.7-fold increase in F4/80 positive macrophages.

6. In Vitro TACE Gene Suppression Effect of Human siTACE/TKPR-9R Complex in Human Macrophage Raw 264.7 Cell Line

(The optimal weight ratio of the TKPR-9R/siRNA complex shown in the subsequent experiment was used as a weight ratio of 4/1 (gene:carrier=1:4).)

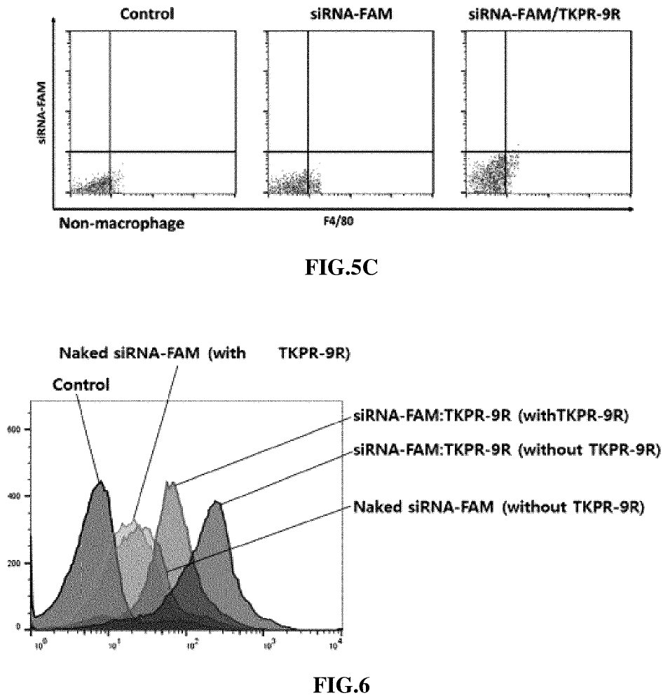

To verify the human macrophage-specific targeting ability of TKPR-9R, competition assay using a free TKPR peptide was performed (FIG. 6).

The experimental process was performed in the same manner as in FIG. 1E. Human monocytes (THP-1) were activated by LPS for 2 hours. After activation, a free TKPR peptide was incubated for 1 hour to block receptors on macrophages. When an NRP-1 receptor was blocked, siRNA alone did not show a change in fluorescence intensity. However, the mean fluorescence intensity (MFI) of the complex significantly decreased when the receptors were occupied by free TKPR. As a result, the cellular uptake of siRNA was mediated by TKPR directly binding to the receptors of the macrophages. Consequently, activated macrophages having NRP-1 receptors in inflammatory tissue may be specifically targeted by the TKPR sequence of the peptide. In addition, the human siTACE/TKPR-9R complex was effectively internalized in the cytoplasm of the THP-1 cells.

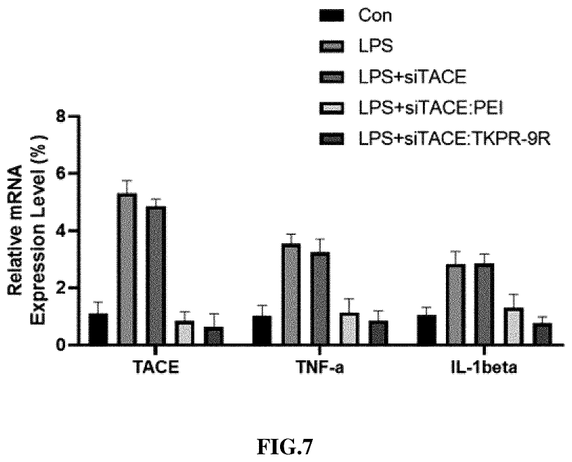

The TKPR-mediated delivery of siTACE showed the suppression of TACE in LPS-induced THP-1 inflammatory macrophages. The transcription of the TACE gene was significantly suppressed (FIG. 7). The expression of TNF-α and IL-1β, which are pro-inflammatory cytokine markers, was also reduced. Accordingly, human siTACE/TKPR-9R may enhance a gene suppression effect in in vivo experiments, compared to siRNA alone.

### CONCLUSION

In previous studies, recruitment of lymphocytes and macrophages in organs damaged by bacterial injection were further exacerbated when pro-inflammatory cytokines such as TNF-α, IL-6 and IL-1β were up-regulated. It was reported that TACE plays a key role in severe inflammatory responses in macrophages in tissue and the peritoneal fluid by triggering excessive conversion of inactive membrane-bound TNF-α to a soluble form.

Accordingly, for treatment of acute inflammatory sepsis, a fundamental therapeutic method that inhibits an inflammatory signaling pathway is essential. Several pharmaceutical companies have tried to develop TACE inhibitor drugs. However, peptide TACE inhibitors showed risks of hepatotoxicity and fibrosis due to the lack of recognition of matrix metalloproteinases (MMPs).

Therefore, a new low-toxicity approach is needed to hinder TACE.

In the present invention, a peptide-based siRNA delivery system was shown to enhance a macrophage-targeting effect by binding to an NRP-1 receptor on the surface of macrophages. Intravenously-injected siRNA-FAM/TKPR-9R and siRNA-Cy5/TKPR-9R complexes exhibited a targeting effect in peritoneal macrophages and tissue-resident macrophages of the lungs, liver, kidneys, and spleen (FIGS. 5A and 5B). The goal was to confirm the siTACE/TKPR-9R complex as a new anti-inflammatory platform for treating sepsis induced by CLP surgery or E. coli and P. aeruginosa bacteria injection. Such results show that the siTACE/TKPR-9R complex down-regulates pro-inflammatory cytokines and chemokines by suppressing the transcription of TACE at the molecular level, and confirmed that the treatment with the complex reduced TACE and cytokine levels in tissue of septic mice. In addition, these results demonstrated that the anti-inflammatory effect of the siTACE/TKPR-9R complex improved a survival rate and histological organ damage. In addition, after the co-administration of two types of antibiotics used for septic patients and the siTACE/TKPR-9R complex, an anti-inflammatory effect and an antibacterial effect were exhibited.

This shows that the siTACE/TKPR-9R complex is effective not only in symptom alleviation but also as a fundamental molecular level solution for sepsis.

In the present invention, the siTACE/TKPR-9R platform aims to find a synergistic therapeutic agent that is able to be co-administered with conventional therapy using an antibiotic to overcome the limitation of monotherapy via an antibiotic, rather than completely replacing an antibiotic. An antibiotic, such as gentamycin, may reduce the amount of bacteria by blocking the synthetic cell membrane of the bacteria, but it is difficult to directly prevent organ damage induced by endotoxins.

Therefore, the present inventors developed a macrophage-targeting therapeutic agent to fundamentally improve an inflammatory pathway mediated by a soluble TNF-α protein while complementing the therapeutic effect of an antibiotic. It was confirmed that the co-administration of the siTACE/TKPR-9R complex and an antibiotic decreases inflammatory responses and damage in organs such as lungs and liver (FIGS. 3C and 3D) and increases a survival rate (FIGS. 3B, 3E, and 3F).

In summary, non-viral gene therapy using the siTACE/TKPR-9R complex was proven to be an innovative sepsis treatment method using macrophage-targeting gene delivery to treat severe inflammatory responses.

The combination treatment of an anti-inflammatory gene therapy and an antibiotic was demonstrated as a promising drug combination for acute and systemic inflammation in organs, serum, and peritoneal fluid.

Consequently, the present invention shows a selective disease cell-targeting effect and an anti-inflammatory effect, overcoming the limitations of antibiotic treatment.

[Sequence Listing Free Text]

[162] <110> Industry-University Cooperation Foundation Hanyang University
[163]
[164] <120> Composite containing gene and gene delivery system for prevent or
[165] treatment of acute inflammatory disease
[166]
[167] <130> P21U10C1311
[168]
[169] <150> KR 10-2021-0073528
[170] <151> 2021-06-07
[171]
[172] <160> 17
[173]
[174] <170> KoPatentIn 3.0
[175]
[176] <210> 1
[177] <211> 19
[178] <212> DNA
[179] <213> Artificial Sequence
[180]
[181] <220>
[182] <223> mouse siTACE Sense sequence
[183]
[184]
[185] <400> 1
[186] acacctgctg caatagtga
19
[187]
[188]
[189] <210> 2
[190] <211> 21
[191] <212> DNA
[192] <213> Artificial Sequence
[193]
[194] <220>
[195] <223> human siTACE Sense sequence
[196]
[197]
[198] <400> 2
[199] gctctcagac tacgatattc t 21
[200]
[201]
[202] <210> 3
[203] <211> 20
[204] <212> DNA
[205] <213> Artificial Sequence
[206]
[207] <220>
[208] <223> mouse siTACE antisense sequence
[209]
[210]
[211] <400> 3
[212] tcactattgc agcaggtgtt
20
[213]
[214]
[215] <210> 4
[216] <211> 21
[217] <212> DNA
[218] <213> Artificial Sequence
[219]
[220] <220>
[221] <223> human siTACE antisense sequence
[222]
[223]
[224] <400> 4
[225] agaatatcgt agtctgagag c
21
[226]
[227]
[228] <210> 5
[229] <211> 15
[230] <212> PRT
[231] <213> Artificial Sequence
[232]
[233] <220>
[234] <223> Cys-TKPR-(9Arg)-Cys
[235]
[236]
[237] <400> 5
[238] Cys Thr Lys Pro Arg Arg Arg Arg Arg Arg Arg Arg Arg Arg Cys
[239] 1 5 10 15
[240]
[241]
[242] <210> 6
[243] <211> 20
[244] <212> DNA
[245] <213> Artificial Sequence
[246]
[247] <220>
[248] <223> mouse TACE Forward primer
[249]
[250]
[251] <400> 6
[252] gtacgtcgat gcagagcaaa
20
[253]
[254]
[255] <210> 7
[256] <211> 20
[257] <212> DNA
[258] <213> Artificial Sequence
[259]
[260] <220>
[261] <223> mouse TACE Reverse primer
[262]
[263]
[264] <400> 7
[265] aaaccagaac agacccaacg
20
[266]
[267]
[268] <210> 8
[269] <211> 23
[270] <212> DNA
[271] <213> Artificial Sequence
[272]
[273] <220>
[274] <223> human TACE forward primer
[275]
[276]
[277] <400> 8
[278] acctgaagag cttgttcatc gag 23
[279]
[280]
[281] <210> 9
[282] <211> 24
[283] <212> DNA
[284] <213> Artificial Sequence
[285]
[286] <220>
[287] <223> human TACE reverse primer
[288]
[289]
[290] <400> 9
[291] ccatgaagtg ttccgataga tgtc 24
[292]
[293]
[294] <210> 10
[295] <211> 24
[296] <212> DNA
[297] <213> Artificial Sequence
[298]
[299] <220>
[300] <223> mouse TNF-alpha forward primer
[301]
[302]
[303] <400> 10
[304] tctcatgcac caccatcaag gact 24
[305]
[306]
[307] <210> 11
[308] <211> 24
[309] <212> DNA
[310] <213> Artificial Sequence
[311]
[312] <220>
[313] <223> mouse TNF-alpha reverse primer
[314]
[315]
[316] <400> 11
[317] accactctcc ctttgcagaa ctca 24
[318]
[319]
[320] <210> 12
[321] <211> 22
[322] <212> DNA
[323] <213> Artificial Sequence
[324]
[325] <220>
[326] <223> human TNF-alpha forward primer
[327]
[328]
[329] <400> 12
[330] ctcttctgcc tgctgcactt tg 22
[331]
[332]
[333] <210> 13
[334] <211> 22
[335] <212> DNA
[336] <213> Artificial Sequence
[337]
[338] <220>
[339] <223> human TNF-alpha reverse primer
[340]
[341]
[342] <400> 13
[343] atgggctaca ggcttgtcac tc 22
[344]
[345]
[346] <210> 14
[347] <211> 22
[348] <212> DNA
[349] <213> Artificial Sequence
[350]
[351] <220>
[352] <223> mouse IL-1beta forward primer
[353]
[354]
[355] <400> 14
[356] tggaccttcc aggatgagga ca
22
[357]
[358]
[359] <210>15
[360] <211> 22
[361] <212> DNA
[362] <213> Artificial Sequence
[363]
[364] <220>
[365] <223> mouse IL-1beta reverse primer
[366]
[367]
[368] <400> 15
[369] gttcatctcg gagcctgtag tg 22
[370]
[371]
[372] <210> 16
[373] <211> 20
[374] <212> DNA
[375] <213> Artificial Sequence
[376]
[377] <220>
[378] <223> human IL-1beta forward primer
[379]
[380]
[381] <400> 16
[382] ggacaagctg aggaagatgc
20
[383]
[384]
[385] <210> 17
[386] <211> 20
[387] <212> DNA
[388] <213> Artificial Sequence
[389]
[390] <220>
[391] <223> human IL-1beta reverse primer
[392]
[393]
[394] <400> 17
[395] tccatatcct gtccctggag
20

1. A gene/carrier complex, comprising:
siRNA that suppresses the expression of tumor necrosis factor-α converting enzyme (TACE); and
a non-viral gene carrier,
wherein the non-viral gene carrier includes tuftsin peptide-oligoarginine.

2. The gene/carrier complex of claim 1, wherein siRNA for TACE has one or more base sequences represented by SEQ ID NO: 1 or 2 and a complementary sequence thereof.

3. The gene/carrier complex of claim 1, wherein the tuftsin peptide-oligoarginine includes cysteines at both ends.

4. The gene/carrier complex of claim 1, wherein the tuftsin peptide-oligoarginine includes Cys-TKPR-(9Arg)-Cys.

5. The gene/carrier complex of claim 1, wherein the tuftsin peptide-oligoarginine targets macrophages.

6. The gene/carrier complex of claim 1, wherein the TACE siRNA and the gene carrier are contained at a weight ratio of 1:2 to 5.

7. A method of preparing a gene/carrier complex, comprising:
mixing siRNA that suppresses the expression of tumor necrosis factor-α converting enzyme (TACE) and a non-viral gene carrier,
wherein the non-viral gene carrier includes tuftsin peptide-oligoarginine.

8. The method of claim 7, wherein the TACE siRNA has one or more base sequences represented by SEQ ID NO: 1 or 2 and a complementary sequence thereof.

9. The method of claim 7, wherein the tuftsin peptide-oligoarginine includes cysteines at both ends.

10. The method of claim 7, wherein the tuftsin peptide-oligoarginine includes Cys-TKPR-(9Arg)-Cys.

11. The method of claim 7, wherein the TACE siRNA and the gene carrier are contained at a weight ratio of 1:2 to 5.

12. The method of claim 7, wherein the incubation is performed at 20 to 40° C. for 20 to 40 minutes.

13. A composition for preventing or treating an acute inflammatory disease, comprising the gene/carrier complex of claim 1 as an active ingredient.

14. The composition of claim 13, wherein the acute inflammatory disease includes one or more selected from the group consisting of sepsis, acute lung injury, acute liver failure, and acute inflammatory bowel disease.

15. The composition of claim 13, wherein the composition is administered orally, by an aerosol, buccally, via the skin, intradermally, by inhalation, intramuscularly, intranasally, intraocularly, intrapulmonarily, intravenously, intraperitoneally, into ears, by injection, by a patch, subcutaneously, sublingually, topically, or transdermally.

16. A method of preventing or treating an acute inflammatory disease, comprising:
administering the composition comprising a therapeutically effective amount of the gene/carrier complex of claim 1 to a subject.

17. The method of claim 16, wherein the acute inflammatory disease includes one or more selected from the group consisting of sepsis, acute lung injury, acute liver failure, and acute inflammatory bowel disease.

18. The method of claim 16, wherein the composition is administered orally, by an aerosol, buccally, via the skin, intradermally, by inhalation, intramuscularly, intranasally, intraocularly, intrapulmonarily, intravenously, intraperitoneally, into ears, by injection, by a patch, subcutaneously, sublingually, topically, or transdermally.
